(19) United States
(12) Patent Application Publication
(10) Pub. No.: US 2023/0117890 A1
(43) Pub. Date: Apr. 20, 2023

(54) INNER STRUCTURE OF A MEDICAL FLUID PUMP

(71) Applicant: B. Braun Melsungen AG, Melsungen (DE)

(72) Inventors: Jan Schwarz, Melsungen (DE); Christoph Erlen, Kassel (DE); Hans-Josef Gerlach, Marsberg (DE); Michael Kauba, Fuldabrueck (DE); Matthias Schwalm, Schwalmstadt (DE); Joachim Schuetz, Fulda (DE); Gerhard Schoeffel, Blaustein (DE); Daniel Wolf, Bibertal (DE)

(21) Appl. No.: 17/968,190

(22) Filed: Oct. 18, 2022

(30) Foreign Application Priority Data
Oct. 19, 2021 (DE) 10 2021 127 062.6

(51) Int. Cl. A61M 5/142

(52) U.S. Cl. CPC A61M 5/14244; A61M 2205/586

(57) ABSTRACT

A medical fluid pump, such as a syringe pump or infusion pump, includes a housing, a front flap hinged to a front side of the housing, and a carrying handle pivotally connected to opposite first and second side surfaces. The carrying handle is pivotable from a storage position to a carrying position in which the carrying handle is disposed above the housing. A plurality of electrical and mechanical components, which are contained within the housing, are necessary for all of the intended functions of the medical fluid pump. A selection of components from the electrical and mechanical components are provided as tare elements, placed within the housing such that a center of gravity of the medical fluid pump is substantially below the carrying handle in the carrying position.

### CROSS-REFERENCE TO RELATED APPLICATIONS

This application claims priority under 35 U.S.C. § 119 to German Application No. 10 2021 127 062.6, filed Oct. 19, 2021, the content of which is incorporated by reference herein in its entirety.

### FIELD

The present disclosure relates to a medical fluid pump and its inner structure.

### BACKGROUND

In medicine, fluid pumps, in particular syringe pumps and peristaltic pumps, are widely used to supply a patient with a defined dose of medication. Since such medical fluid pumps are expensive medical equipment, not every patient station is usually equipped with such a medical fluid pump, but rather the medical fluid pump is installed at the patient station as needed. For this reason, such medical fluid pumps are often mobile or partially mobile devices that are brought by a practitioner to the respective patient or patient site in question and are installed there. This also has the advantage that if the patient is transferred to another ward or an operating room, the patient does not have to be disconnected from the medical fluid pump, thus ensuring a consistent, uninterrupted supply of the defined dose of medication to the patient.

In order to ensure easy handling and easy transport for the practitioner, the medical fluid pump may be equipped with a carrying handle.

It has now been shown that when the medical fluid pump is transported on the carrying handle, tilting of the housing of the medical fluid pump from a horizontal position can occur. This is particularly problematic if the patient continues to be supplied by the medical fluid pump and the tilting may impair the proper function of the medical fluid pump.

In order to solve this problem, various solutions were considered. The carrying handle was locked in a carrying position relative to the housing so that tilting of the housing relative to the carrying handle in the carrying position could be prevented. However, this had the disadvantage that further moving components in the form of the lock had to be provided on the medical fluid pump, which on the one hand are difficult to clean and can thus represent a breeding ground for germs and bacteria, and on the other hand represent a further component that makes the medical fluid pump more expensive and represents a further source of damage to the medical fluid pump.

In a further approach, pivot points of the carrying handle were designed to be movable in order to make the carrying position variable, so that the carrying handle could be moved in the carrying position over a center of gravity of the fluid pump. Here, too, however, it was found that the additional movable components in the form of a slidable bearing made the medical fluid pump more expensive, represented a further source of damage and made handling of the medical fluid pump significantly more difficult.

A third approach was to provide metallic, in particular lead, counterweights, which were placed in the housing of the medical fluid pump in such a way that a center of gravity of the medical fluid pump is located below the carrying handle in the carrying position. However, the insertion of counterweights resulted in an increase in the overall weight of the medical fluid pump, making it more difficult for practitioners to handle.

### SUMMARY

The objects and aims of the disclosure are to eliminate or at least reduce the disadvantages of the prior art and in particular to provide a medical fluid pump having a carrying handle, wherein tilting of a housing against the carrying handle is prevented in a carrying position. In particular, it is to be achieved that in a carrying position, the housing of the fluid pump is oriented substantially horizontally without resulting in the disadvantages explained above.

The medical fluid pump, in particular in the form of an infusion pump or syringe pump, comprises a housing, a front flap hinged to a front side of the housing, a carrying handle pivotably hinged to opposite first and second side surfaces and pivotable from a storage position to a carrying position in which it is positioned above the housing, and a number of electrical and mechanical components within the housing required for all intended functions of the medical fluid pump. A selection of components from the electrical and mechanical components within the housing are provided as tare elements positioned within the housing such that the center of gravity of the fluid pump is substantially below the carrying handle in the carrying position.

In other words, the medical fluid pump, which is preferably configured as a syringe or peristaltic pump, includes the housing with a front flap hinged to the front side of the housing via hinges, which preferably has a display in the form of a touch display. The carrying handle, which preferably has a bracket shape, is hinged to the two side surfaces that extend from the outer rims of the front side of the housing in the direction of the rear side of the housing. The carrying handle can thus be folded from a folded position, in which the carrying handle rests against the housing, to a carrying position, in which the carrying handle is located above the housing. The electrical and mechanical components of the mechanical fluid pump, which are necessary for the implementation of all functions, in particular technical functions, are located within the housing. Of these electrical and mechanical components, a number of components are selected and arranged in the housing in such a way that the center of gravity is substantially in a vertical plane in which the carrying handle is also located in the carrying position. As a result, the housing is suspended from the carrying handle in a substantially horizontal position or orienting itself substantially horizontally, respectively.

The core of the invention is therefore that a selection of the electrical and mechanical components that are necessary to implement the objects the medical fluid pump has to fulfil are used as balance or tare weights. In this way, it can be ensured that the medical fluid pump or the housing of the medical fluid pump is brought into a desired position/orientation in relation to an environment when it is suspended from the carrying handle, without the need for additional components such as (lead) weights or engaging hinges or the like.

The electrical and/or mechanical components may all be components required for the function of the medical fluid pump.

This is also desirable, since several of the medical fluid pumps may be suspended below each other, and can then be carried by the carrying handle of the uppermost medical fluid pump. If the centers of gravity of the medical fluid pumps suspended one below the other are substantially in one plane with the carrying handle in the carrying position, comfortable carrying is possible. If, on the other hand, the centers of gravity of the medical fluid pumps suspended one below the other are not below the carrying handle, this leads to an unergonomic carrying position, which may result in rapid fatigue of the carrier.

In a first aspect, the selection of components from the electrical and mechanical components may include at least one energy storage device and/or a power adaptor.

In other words, at least the energy storage device and/or the power adaptor may be used as tare weights. In yet other words, the position of the center of gravity of the medical fluid pump can be influenced by the suitable placement of the energy storage device and/or the power adaptor so that the center of gravity is substantially below the carrying handle in the carrying position. Since the energy storage device, which is configured in particular as a rechargeable battery, for example in the form of a lead-acid battery, a nickel-metal hydride battery, a lithium-ion battery, a lithium-polymer battery or a lithium-metal battery, and the power adaptor are components that have a high specific weight due to the materials used in them, but are relatively flexible in their placement within the medical fluid pump, these components are particularly suitable for specifically manipulating or influencing the center of gravity of the medical fluid pump.

In a further aspect, the selection of components from the electrical and mechanical components may include at least a drive train and/or a motor.

In other words, at least the drive train and/or the motor can be used as tare weights. In yet other words, by appropriate placement of the drive train and/or the motor, the position of the center of gravity of the medical fluid pump can be influenced such that the center of gravity is substantially below the carrying handle in carrying position. Since the drive train, which is configured in particular as a holding arm with a drive head that can be moved linearly with respect to the housing or as a peristaltic system, and the motor that drives the drive train are components that have a high specific weight and also a high absolute weight due to the materials used in them, these components are suitable for specifically manipulating or influencing the center of gravity of the medical fluid pump.

In a further aspect, a first assembly, preferably in the form of the drive train, may be configured in a first longitudinal half of the housing facing the front side of the housing, and a second assembly, preferably in the form of the power adaptor and/or of a communication module may be configured in a second longitudinal half of the housing facing the rear side of the housing opposite the front side of the housing, wherein a weight of the first assembly balances a weight of the second assembly with respect to a tilt axis through the pivot points.

In other words, the first assembly, which in particular may be the drive train, may be arranged inside the housing in the first longitudinal half, which is directly adjacent to the front flap. The second assembly, which in particular may be the power adaptor and/or the communication module, may be arranged inside the housing in the second longitudinal half, which is arranged between the rear side of the housing and the first longitudinal half. A first torque generated by a first weight force of the first assembly with respect to the tilt axis through the pivot points of the carrying handle is substantially cancelled by a second torque generated by a second weight force of the second assembly with respect to the tilt axis through the pivot points of the carrying handle. In yet other words, there is substantially a torque equilibrium of the first weight force and the second weight force, relative to the tilt axis.

In a further aspect, a third assembly, which may preferably be configured in the form of the drive head outside the housing on the second side surface, may be configured to move linearly relative to the housing via a drive arm, and a fourth assembly, preferably in the form of the motor, may be arranged in a first transverse half of the housing facing away from the drive head.

In other words, the third assembly, which is preferably the drive head that is linearly movable to the housing via the holding arm, may be configured outside of the housing of the medical fluid pump adjacent to the second side surface. The fourth assembly, which in particular may be configured as the motor responsible for driving the drive head, may be arranged in the first transverse half of the interior space of the housing adjacent to the first side surface and spaced from the second side surface.

In yet other words, the interior space of the housing may be divided into a first transverse half and a second transverse half. The first transverse half is adjacent to the first side surface and to the second transverse half and includes the fourth assembly, which in particular is the motor. The second transverse half is adjacent to the second side surface and the first transverse half. A third assembly, preferably in the form of the drive head, may be arranged outside the housing, adjacent to the second side surface which bounds the second transverse half. In this way, the third assembly and the fourth assembly are arranged as far away from each other as possible. On the one hand, this allows the fourth assembly to act as a kind of counterweight for the third assembly, in particular when the third assembly is extended. On the other hand, this effectively prevents vibrations that occur in the fourth assembly from being transmitted to the third assembly and impairing its function.

In a further aspect, the energy storage device may be arranged substantially in the center of gravity of the medical fluid pump.

In other words, the energy storage device is disposed substantially below the carrying handle in the carrying position. Since the energy storage device is a component with a high specific and absolute weight, it is advantageous to arrange it in the desired center of gravity of the medical fluid pump.

In a further aspect, the communication module may be configured/connected to a first antenna and a second antenna, wherein the first antenna is configured at the first side surface and the second antenna is configured at the second side surface.

In other words, the medical fluid pump may include the communication module, which may have two external antennas. The first antenna is preferably fixed to the first side surface in a first antenna receptacle and the second antenna is fixed to the second side surface in a second antenna receptacle. Such an arrangement of the antennas can prevent possible interference of the transmission power of the antennas with each other.

In a further aspect, the distance between the first side surface and the second side surface may be matched to the transmission frequency of the first antenna and the second antenna. Preferably, the distance corresponds to substantially a multiple of a quarter, and more preferably a multiple of a half, of the wavelength of the transmission frequency.

In other words, the transmission frequency of the antennas is 2.4 GHz and/or 5 GHz. The wavelength is 12.5 cm for 2.4 GHz and 6 cm for 5 GHz. The distance between the antennas and thus between the first side surface and the second side surface is thus a multiple of 3.125 cm or a multiple of 3 cm, respectively.

In yet other words, the dimensions, in particular a width dimension, of the medical fluid pump are predetermined by the transmission frequency of the antennas of the communication module. For this reason, the dimensions of the medical fluid pump cannot be changed at will, but are substantially predetermined.

In a further aspect, the fourth assembly, preferably in the form of the motor, may be arranged in the first transverse half of the housing and a fifth assembly, preferably in the form of a deadbolt drive, may be arranged in the second transverse half of the housing different from the first transverse half.

In other words, the fourth assembly, preferably in the form of the motor, and the fifth assembly, preferably in the form of the deadbolt drive, are arranged in different transverse halves of the interior space of the housing of the medical fluid pump. In this way, the fourth assembly and the fifth assembly are arranged as far away from each other as possible. In this way, vibrations that occur in the fourth assembly or in the fifth assembly can be effectively prevented from being transmitted to the other one of the fifth assembly of the fourth assembly, respectively, and from impairing their function.

### BRIEF DESCRIPTION OF THE DRAWINGS

FIG. 1 is a perspective view of a medical fluid pump according to the disclosure, shown here as an infusion pump or peristaltic pump, respectively.

FIG. 2 is a side view of a medical fluid pump according to the disclosure, shown here as an infusion pump or peristaltic pump, respectively.

FIG. 3 is a perspective view of three medical fluid pumps according to the disclosure, which are carried in a combination by an operator.

FIG. 4 is an illustration of an interior space of a medical fluid pump in the form of a syringe pump.

FIG. 5 is an illustration of the interior space of a medical fluid pump in the form of an infusion pump or peristaltic pump, respectively.

The Figures are schematic in nature and serve only to aid understanding of the disclosure. Identical elements are marked with the same reference signs.

### DETAILED DESCRIPTION

Preferred embodiments are described below on the basis of the Figures. Here, features and characteristics are described by way of example for individual embodiments. It should be explicitly mentioned that features described here are not limited to the respective embodiment.

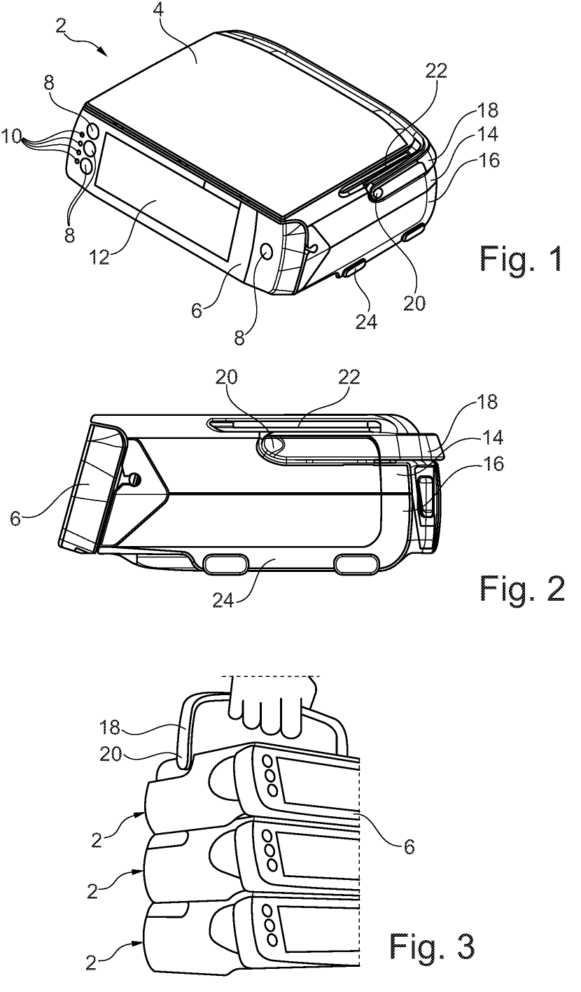

FIG. 1 shows a medical fluid pump 2 according to a preferred embodiment. The medical fluid pump 2 shown in FIG. 1 is designed as an infusion pump. Naturally, the medical fluid pump may also be configured as a syringe pump. As shown in FIG. 1, the medical fluid pump 2 has a substantially cuboid housing 4, on the front side of which is a front flap 6 pivotably hinged to the housing 4. A plurality of operating buttons 8, a plurality of signal lights 10, and a touch display 12 are arranged on a front side of the front flap 6.

The housing 4 further comprises an upper housing shell 14 and a lower housing part 16, which can be connected to each other and thus form the housing 4. A carrying handle 18 in the form of a handle bar is pivotably hinged to the upper housing shell 14. The carrying handle 18 embraces the upper housing shell 14 in a width direction, so that the carrying handle 18 is hinged to both side surfaces or to a first side surface and a second side surface of the upper housing shell 14 or of the housing 4, respectively. The carrying handle 18 is connected to the housing 4 at pivot points 20. The pivot points 20 are configured as hinges and are located, as shown in FIG. 1 and FIG. 2, substantially centrally in a longitudinal pump direction. The longitudinal pump direction is to be understood as a direction starting from the front side with the front flap 6 to a rear side of the medical fluid pump 2 opposite the front side. When the carrying handle 18 is folded out from the storage position into a carrying position, the carrying handle pivots about the pivot points 20. The axis of rotation thus passes through the pivot points 20.

FIG. 2 is an illustration of a side view of the medical fluid pump 2. In the upper housing shell, upper latching rails 22 are configured in the first side surface and the second side surface. In the lower housing shell, lower latching rails 24 are configured in the first side surface and the second side surface. The lower latching rails 24 of a first medical fluid pump 2 are provided and configured to engage the upper latching rails of a second, identically-constructed medical fluid pump 2 and to couple the first medical fluid pump 2 and the second medical fluid pump 2 together.

In FIG. 1 and FIG. 2, the medical fluid pump is shown in a state in which the carrying handle 18 is folded in and rests against the upper housing shell 14. In the folded state, the carrying handle 18 is flush with the side surfaces.

As shown in FIG. 3, according to the preferred configuration example, three medical fluid pumps 2 can be stacked on top of each other, connected/coupled together, and can be carried by the carrying handle 18 of the uppermost medical fluid pump 2. That is, the carrying handle 18 of the medical fluid pump 2 is configured and dimensioned to carry a combination of three medical fluid pumps 2 coupled together. The carrying handle 18 of the uppermost medical fluid pump 2 shown in FIG. 3 is in the carrying position. According to the invention, a respective center of gravity of the medical fluid pumps is in a substantially vertically arranged plane with the carrying handle 18. The housing 4 is oriented substantially horizontally in the carrying position. The carrying handle 18 is oriented substantially vertically in the carrying position.

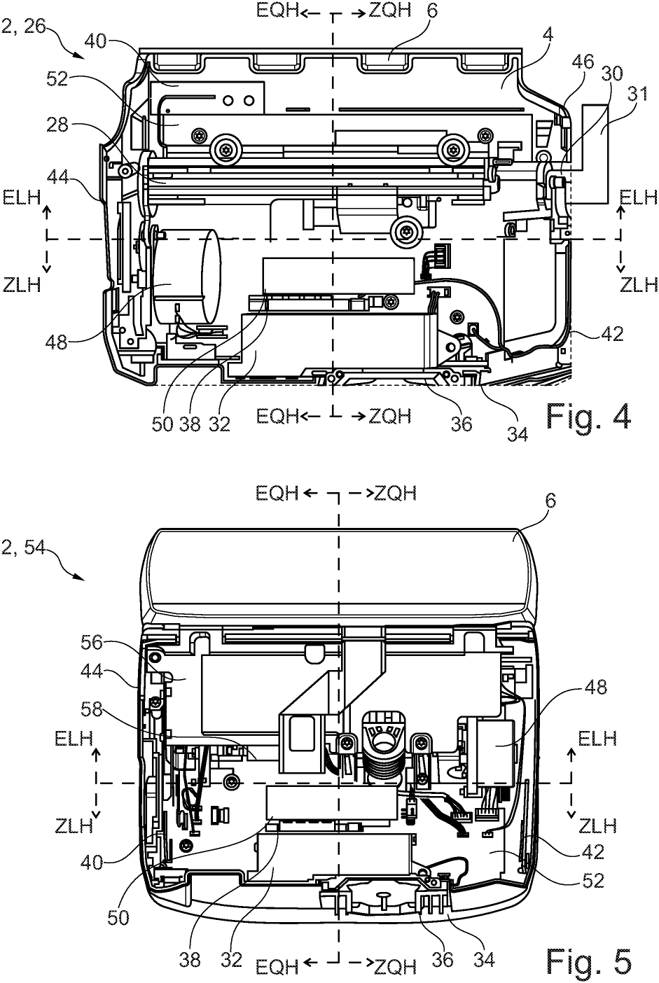

FIG. 4 shows the inner structure of the medical fluid pump 2 according to the disclosure in the form of a syringe pump 26. The syringe pump 26 includes a drive train 28, which is provided and configured to include a holding arm 30, at the end of which a drive head 31 is provided that can be moved linearly with respect to the housing 4. The drive train 28 is configured in a first longitudinal half ELH of the housing 4 facing the front side of the housing and thus the front flap 6. A power adaptor 32 is configured in a second longitudinal half ZLH of the housing 4. The second longitudinal half faces a rear side 34 opposite the front flap 6. Advantageously, the power adaptor 32 is configured to abut the rear side 34. This enables cooling ribs 36 to be provided on the rear side 34 of the housing 4. These cooling ribs 36 are provided and configured to cool the power adaptor 32. In addition to the power adaptor 32, a wireless communication module 38 is also provided and configured in the second longitudinal half ZLH. In particular, the wireless communication module 38 is arranged between the power adaptor 32 and the drive train 28. In other words, the wireless communication module 38 is arranged between the rear side 34 and the first longitudinal half ELH. The wireless communication module 38 is connected to a first antenna 40 and to a second antenna 42, wherein the first antenna 40 is provided and configured adjacent to a first side surface 44 of the housing 4. The second antenna 42 is provided and configured adjacent to a second side surface 46 of the housing 4. The first antenna 40 and the second antenna 42 are oriented orthogonally to each other. The distance between the first side surface 44 and the second side surface 46 corresponds to a multiple of a quarter or a multiple of a half wavelength, respectively, of the transmission frequency of the first antenna 40 and the second antenna 42. A motor 48 driving the drive train 28 is configured in a first transverse half EQH of the housing 4. The drive head 31 is adjacent to the second transverse half ZQH of the housing 4. An energy storage device 50 is configured between the wireless communication module 38 and the drive train 28. A main board 52 is disposed in a planar manner within the housing 4. The main board 52 includes substantial control devices for the syringe pump 26.

FIG. 5 shows an inner structure of the medical fluid pump 2 in the form of a further preferred embodiment. More precisely, FIG. 5 shows the inner structure of the medical fluid pump 2 according to the disclosure of the infusion pump 54 or peristaltic pump 54 type. The infusion pump 54 includes a drive train 56 in the form of a peristaltic system, which is provided and configured to convey a medical fluid via a peristaltic system. The drive train 56 is configured in the front side of the housing, and thus in the first longitudinal half ELH of the housing 4 facing the front flap 6. The power adaptor 32 is configured in the second longitudinal half ZLH of the housing 4. The second longitudinal half ZLH faces the rear side 34 opposite the front flap 6. Advantageously, the power adaptor 32 is configured to abut the rear side 34. This allows cooling ribs 36 to be provided on the rear side 34 of the housing 4. These cooling ribs 36 are provided and configured to cool the power adaptor 32. In addition to the power adaptor 32, a wireless communication module 38 is also provided and configured in the second longitudinal half ZLH. In particular, the wireless communication module 38 is arranged between the power adaptor 32 and the first longitudinal half ELH. The wireless communication module 38 is connected to the first antenna 40 and to the second antenna 42, wherein the first antenna 40 is provided and configured adjacent to the first side surface 44 of the housing 4. The second antenna 42 is provided and configured adjacent to the second side surface 46 of the housing 4. The first antenna 40 and the second antenna 42 are oriented orthogonally to each other. The distance between the first side surface 44 and the second side surface 46 is a multiple of a quarter or a multiple of a half wavelength, respectively, of the transmission frequency of the first antenna 40 and the second antenna 42. The motor 48, which drives the drive train 56, is configured in the second transverse half ZQH of the housing 4. A deadbolt 58, is configured in the first transverse half EQH of the housing 4. An energy storage device 50 is configured between the wireless communication module 38 and the drive train 56. The main board 52 is disposed in a planar manner within the housing 4. The main board 52 includes essential control devices for the infusion pump 54.

What is claimed:

1. A medical fluid pump comprising:
a housing;
a carrying handle pivotably hinged to the housing and pivotable from a storage position to a carrying position in which it is arranged above the housing; and
a number of electrical and mechanical components within the housing which perform intended functions of the medical fluid pump,
wherein a selection of components from the electrical and mechanical components within the housing are provided as tare elements positioned within the housing such that a center of gravity of the medical fluid pump is substantially below the carrying handle in a carrying position.

2. The medical fluid pump according to claim 1, wherein the selection of components from the electrical and mechanical components includes at least an energy storage device and/or a power adaptor.

3. The medical fluid pump according to claim 1, wherein the selection of components from the electrical and mechanical components includes at least a drive train and/or a motor.

4. The medical fluid pump according to claim 1, wherein a first assembly is configured in a first longitudinal half of the housing facing the front side of the housing and a second assembly is configured in a second longitudinal half of the housing facing the rear side of the housing opposite the front side of the housing, wherein a weight of the first assembly balances a weight of the second assembly with respect to an axis of rotation through the pivot points.

5. The medical fluid pump according to claim 1, wherein a third assembly, which is configured in the form of a drive head outside the housing on the second side surface of the housing to move linearly relative to the housing via a drive arm, and a fourth assembly in the form of the motor is arranged in a first transverse half of the housing facing away from the drive head.

6. The medical fluid pump according to claim 1, wherein the energy storage device is arranged substantially in the center of gravity of the medical fluid pump.

7. The medical fluid pump according to claim 4, wherein the communication module is connected/configured with a first antenna and a second antenna, wherein the first antenna is configured at the first side surface of the housing and the second antenna is configured at the second side surface of the housing.

8. The medical fluid pump according to claim 7, wherein a distance between the first side surface and the second side surface is matched to the transmission frequency of the first and second antennas.

9. The medical fluid pump according to claim 1, wherein the fourth assembly in the form of the motor is arranged in a second transverse half of the housing different from the first transverse half, and a fifth assembly in the form of a deadbolt drive is arranged in the first transverse half of the housing.

10. The medical fluid pump according to claim 1, wherein the medical fluid pump is a syringe or infusion pump.

11. The medical fluid pump according to claim 4, wherein the first assembly is a drive train and the second assembly is a power adapter and/or a communication module.

12. The medical fluid pump according to claim 8, wherein the distance corresponds to a multiple of a quarter of the transmission frequency.

13. The medical fluid pump according to claim 8, wherein the distance corresponds to a multiple of a half of the transmission frequency.
